(19) United States
(12) Patent Application Publication
(10) Pub. No.: US 2009/0206280 A1
(43) Pub. Date: Aug. 20, 2009

(54) CHARGED-BEAM EXPOSURE APPARATUS HAVING AN IMPROVED ALIGNMENT PRECISION AND EXPOSURE METHOD

(76) Inventors: Takeshi KOSHIBA, Yokohama-shi (JP); Tetsuro NAKASUGI, Yokohama-shi (JP); Ryoichi INANAMI, Yokohama-shi (JP); Takumi OTA, Yokohama-shi (JP); Hiroyuki MIZUNO, Yokohama-shi (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/363,123

(22) Filed: Jan. 30, 2009

(30) Foreign Application Priority Data
Jan. 31, 2008 (JP) 2008-021998

(51) Int. Cl. A61N 5/00; H01J 3/14

(52) U.S. Cl. 250/492.2; 250/396 R

(57) ABSTRACT

The first charged-beam optical system, which is one of the charged-beam optical systems, detects first marks provided on the chips formed in the wafer. The positions of the chips made in the wafer are calculated from position data about the first marks detected. The charged-beam optical systems detect the second mark provided on a stage. The position of the beam generated by each charged-beam optical system is adjusted in accordance with position data about the second mark detected. The charged-beam optical systems are used in accordance with the positions of the chips, to thereby draw a pattern.

### CROSS-REFERENCE TO RELATED APPLICATIONS

This application is based upon and claims the benefit of priority from prior Japanese Patent Application No. 2008-021998, filed Jan. 31, 2008, the entire contents of which are incorporated herein by reference.

### BACKGROUND OF THE INVENTION

1. Field of the Invention

The present invention relates to a charged-beam exposure apparatus that has, for example, a plurality of charged-beam optical systems, and also to an exposure method.

2. Description of the Related Art

Any electron-beam exposure apparatus that applies a charged beam, such as an electron beam, to a substrate, thereby forming a pattern on the substrate, is advantageous in that it they can form fine patterns. Since the electron-beam exposure apparatus scans the substrate with an electron beam to form a specific pattern, the exposure time is longer than in exposure apparatuses that apply light to a substrate at a time, such as steppers and X-ray exposure apparatus. The electron-beam exposure apparatus has but a small throughput. This is one problem with the electron-beam exposure apparatus.

Further, to form a fine pattern, the Coulomb repulsion of the electron beam needs to be suppressed. To suppress the Coulomb repulsion, the beam current must be reduced. If the beam current is reduced, a long time is spent to achieve the exposure for forming the fine pattern, inevitably decreasing the throughput of the electron beam exposure. This is another problem with the electron-beam exposure apparatus.

To solve the two problems specified above, multibeam exposure apparatuses having a plurality of electron-beam optical systems have been proposed (see, for example, Jpn. Pat. Appln. KOKAI Publication No. 2004-15069). In the multibeam exposure apparatus disclosed in this publication, the beam optical systems can draw patterns in a plurality of regions on the surface of the wafer, almost at the same time. This can increase the throughput. The multibeam exposure apparatus is, however, disadvantageous in the following respect.

If the electron-beam optical systems perform global alignment to adjust the distortion in the surface of the wafer, a complicated calculation is performed to detect the positions of the chips formed in the wafer. The complicated calculation may result in errors in detecting the positions of chips. The position error of the beam applied by each electron-beam optical system must be obtained and added to the position of the mark provided on each chip, which has been determined by the electron-beam optical system. Therefore, the mark position determined by the electron-beam optical system is corrected by using the detection error of the beam position. This correction results in another error, making it difficult to increase the alignment precision. In view of this, there is a demand for a charged-beam exposure apparatus and charged-beam exposure method, which use a plurality of electron-beam optical systems and which can enhance the alignment precision.

### BRIEF SUMMARY OF THE INVENTION

According to a first aspect of the invention, there is provided a charged-beam exposure apparatus comprising: a plurality of charged-beam optical systems; a stage configured to move with respect to the charged-beam optical systems and to hold a wafer; and a control unit configured to control the charged-beam optical systems and stage. The control unit controls the first charged-beam optical system, which is one of the charged-beam optical systems, to detect first marks provided on chips formed in the wafer. The control unit calculates positions of the chips made in the wafer, from position data about the first marks detected. The control unit controls the charged-beam optical systems to detect a second mark provided on the stage. The control unit adjusts position of the beam generated by each charged-beam optical system, in accordance with position data about the second mark detected.

According to a second aspect of the invention, there is provided a charged-beam exposure method comprising: detecting first marks provided on chips formed in a wafer, by using first charged-beam optical system, which is one of charged-beam optical systems; calculating positions of the chips made in the wafer, from position data about the first marks detected; detecting a second mark provided on a stage holding the wafer; and adjusting position of the beam generated by each charged-beam optical system, in accordance with position data about the second mark detected.

### BRIEF DESCRIPTION OF THE SEVERAL VIEWS OF THE DRAWING

FIG. 1 is a flowchart explaining how an embodiment operates;

FIG. 2 is a diagram showing the configuration of an electron-beam exposure apparatus according to the embodiment;

FIG. 3 is a diagram schematically showing the relationship the beam-deflection region of each column has with the wafer in the electron-beam exposure apparatus according to the embodiment; and

FIG. 4 is a plan view showing a wafer in which chips are formed.

### DETAILED DESCRIPTION OF THE INVENTION

An embodiment of this invention will be described with reference to the accompanying drawings.

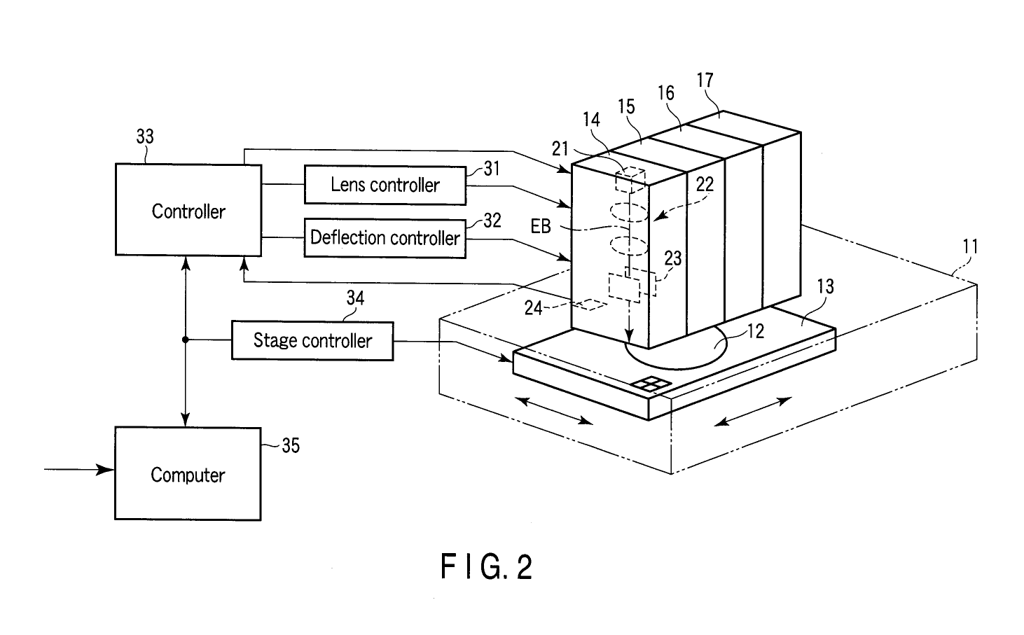

FIG. 2 shows the configuration of an electron-beam exposure apparatus according to an embodiment. The apparatus has a chamber 11 and stage 13. The stage 13 is provided in the chamber 11, and holds a wafer 12. The stage 13 can move in the chamber 11, in X-direction and Y-direction.

On the chamber 11, a plurality of columns 14 to 17 are arranged, each constituting an electron-beam optical system. For simplicity of explanation, four columns are shown in FIG. 2. Nonetheless, more columns may be provided. Each of columns 14 to 17 contains an electron gun 21, plurality of lenses 22, deflector 23, and reflection electron detector 24. The electron gun 21 generates an electron beam EB. The electron beam EB has its diameter reduced while passing through the lenses 22, and is deflected by the deflector 23 and is applied to the wafer 12. Thus, the electron beam EB scans the wafer 12, drawing a pattern on the wafer 12. As will be described later, the reflection electron detector 24 detects the electrons constituting the beam reflected from the surface of the wafer 12 or stage 13.

The electron-beam exposure apparatus further has a lens controller 31, deflection controller 32, controller 33, stage controller 34, and computer 35. The lens controller 31 controls the lenses 22 of columns 14 to 17. The deflection controller 32 controls the deflector 23. The lens controller 31, deflection controller 32, the electron gun 21 of each column, and the reflection electron detector 24 of each column are connected to the controller 33. The controller 33 and stage controller 34 are connected to the computer 35. The computer 35 controls the controller 33 and stage controller 34.

Columns 14 to 17 are allocated to exposure regions of the wafer 12, respectively.

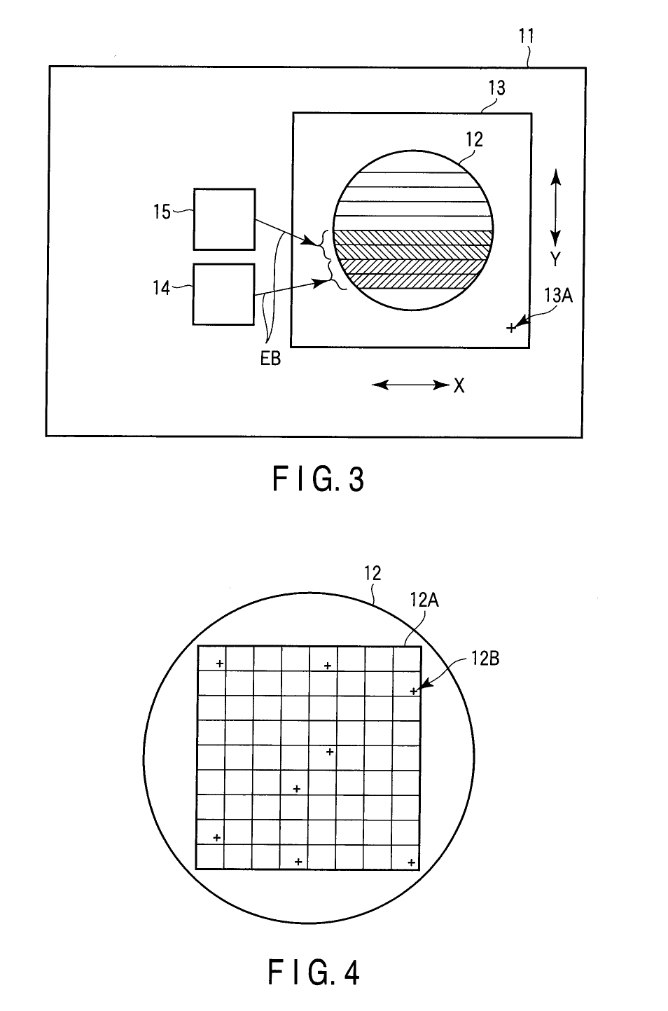

FIG. 3 is a diagram schematically showing the relationship columns 14 and 15 have with the beam-deflection region of the wafer 12. Note that the positions columns 14 and 15 take in FIG. 3 differ from the positions they actually assume in the electron-beam exposure apparatus. In FIG. 3, columns 16 and 17 are not illustrated.

As FIG. 3 shows, the wafer 12 has drawing regions, which are allocated to columns 14 and 15. Each of columns 14 and 15 applies an electron beam EB to a chip provided on the wafer 12 while the drawing region allocated to it remains in the beam-deflection region for the column after the stage 13 has been moved. The beam-deflection region has a size of, for example, 100 microns to several millimeters. When drawing in a range exceeding the deflection region, the stage 13 is moved so that the drawing region may be located in the beam-deflection region.

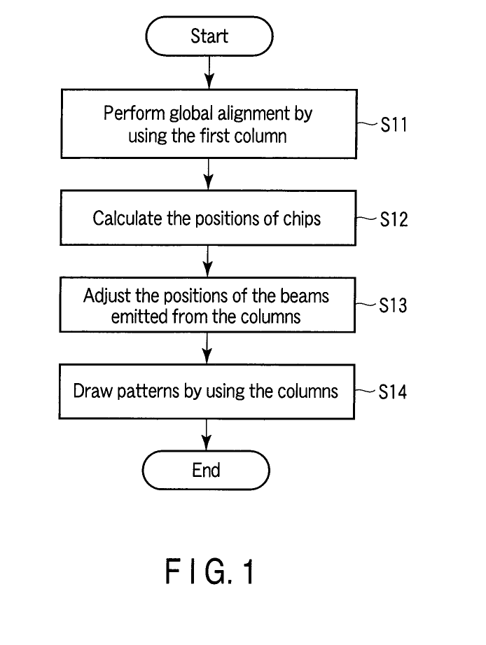

How the configuration described above performs electron-beam exposure will be explained with reference to FIGS. 1 to 4.

First, the global alignment of the wafer 12 is performed, using one of columns 14 to 17 (S11). As shown in FIG. 4, an alignment mark 12B is made on each chip 12A formed in the wafer 12. The alignment mark 12B is, for example, a cross and has been formed by etching the silicon substrate (wafer 12). Only some of the alignment marks 12B are illustrated in FIG. 4. In the global alignment, some alignment marks 12B selected beforehand are detected. These are, for example, the alignment marks 12B made on eight representative chips.

More specifically, the stage 13 is moved in the X-direction and Y-direction, bringing the alignment mark 12B of a selected chip 12A into the beam-deflection region of column 14. Column 14 applies an electron beam EB to the alignment mark 12B. The alignment mark 12B reflects the electron beam EB. The electrons constituting the beam EB reflected are detected by the detector 24 of column 14. The electrons are supplied, the form of an electron signal, from column 14 to the controller 33. The controller 33 measures the level of the electron signal, determining the position of the alignment mark 12B. This process sequence is repeated for the other chips. As a result, the alignment marks of eight chips 12A are detected.

Further, a sensor (not shown) extending in Z-direction detects the heights of the chips 12A formed in the wafer 12. The data representing the heights of the chips 12A is supplied to the controller 33.

From the positions of the eight alignment marks 12B and data generated by the Z-direction sensor, the controller 33 determines whether the wafer is distorted and whether the chips 12A are distorted over the entire surface of the wafer 12.

Thereafter, the positions of all chips made in the wafer 12 are calculated from the result of the global alignment (S12).

Next, the beams emitted from columns 14, 15, 16 and 17 are adjusted in position (S13). This adjustment of the beam positions is accomplished by using columns 14, 15, 16 and 17. As shown in FIG. 3, an alignment mark 13A is made in the surface of the stage 13 at a prescribed position. This alignment mark 13A has the same shape as the alignment marks 12B, and is made on the chips 12A in the same way as the alignment marks 12B are made on the chips 12A.

In adjusting the beam positions, columns 14 to 17 are used, detecting the alignment mark 13A in the same way as the alignment marks 12B are detected, and the offset between any two adjacent columns, i.e., positional deviation of the columns, is adjusted. More precisely, the stage 13 is moved in the X-direction and Y-direction, bringing the alignment mark 13A into the beam-deflection region of one column. While the alignment mark 13A into the beam-deflection region of the column. In this state, the column applies an electron beam to the alignment mark 13A, and the detector 24 of the column detects the electron beam reflected. The detector 24 generates a signal from the electrons. The signal is supplied to the controller 33. From the level of the signal, the controller 33 determines the position of the position of the alignment mark 13A. This process sequence is repeated, whereby the other columns detect the alignment mark 13A.

From the detection signals supplied from columns 14 to 17, the controller 33 calculates the offsets, each between two electron beam emitted from two adjacent ones of columns 14 to 17. Each offset thus calculated is added to the distance between the two adjacent columns (i.e., distance between two adjacent electron beams), which distance is known. That is, the distance between columns 14 and 15 is known, and the offset of the beam emitted from column 15 is added to that distance, thereby eliminating the positional deviation of column 15. Similar calculation is repeated for the other columns, thereby eliminating the positional deviations of the other columns.

After the positions of the beams emitted from all columns have been adjusted as described above, columns 14 to 17 draw prescribed patterns on the chips, respectively, in accordance with the chip positions calculated in Step S12 (S14).

In the embodiment described above, the global alignment is performed, using column 14, i.e., one of the four columns 14 to 17. The positions of all chips made in the wafer 12 are then calculated from the result of the global alignment. The chip positions can therefore be easily calculated within a short time.

Moreover, since only one column (e.g., column 14) is used to calculate the position of all chips, the chip-position calculation error can be smaller than in the case where a plurality of columns are used to calculate the chip positions.

As described above, columns 14 to 17 detect the alignment mark 13A, and the positions of the beams emitted from columns 14 to 17 are adjusted. Therefore, the beam emitted from any column can be adjusted with high precision in the electron-beam exposure apparatus.

Thus, the position of each chip can be calculated to high accuracy, and the positions of the beams emitted from columns 14 to 17 can be adjusted with high precision. The electron-beam exposure apparatus can therefore draw fine and high-precision patterns.

Further, the position of beam emitted from each column is adjusted immediately before a pattern is drawn. This ensures high-precision drawing of patterns. That is, the electron beam emitted from each column drifts by a distance in the order of, for example, nanometer, because electrons accumulate in the insulating member of the deflection system. The drift of the beam can be minimized by adjusting the positions of the beams emitted from the columns, immediately before the drawing of a pattern is started. Hence, a pattern can be drawn with high precision.

The embodiment described above is an electron-beam exposure apparatus. The present invention is not limited to an electron-beam exposure apparatus, nevertheless. The invention can be applied to an exposure apparatus that applies an ion beam as a charged beam.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A charged-beam exposure apparatus comprising:
a plurality of charged-beam optical systems;
a stage configured to move with respect to the charged-beam optical systems and to hold a wafer; and
a control unit configured to control the charged-beam optical systems and stage,
wherein the control unit controls the first charged-beam optical system, which is one of the charged-beam optical systems, to detect first marks provided on chips formed in the wafer, calculates positions of the chips made in the wafer, from position data about the first marks detected, controls the charged-beam optical systems to detect a second mark provided on the stage, and adjusts position of the beam generated by each charged-beam optical system, in accordance with position data about the second mark detected.

2. The apparatus according to claim 1, wherein the control unit draws a pattern by using the charged-beam optical systems in accordance with the positions of the chips.

3. The apparatus according to claim 2, wherein the control unit adjusts the position of the beam generated by each charged-beam optical system, immediately before using the charged-beam optical systems to draw a pattern.

4. The apparatus according to claim 3, wherein the control unit adjust the position of the beam generated by each charged-beam optical system, in accordance with a distance between the first charged-beam optical system and a second charged-beam optical system, and with position data about the second mark detected.

5. The apparatus according to claim 3, wherein each of the charged-beam optical systems comprises:
a beam generator configured to generate a charged beam;
a deflector configured to deflect the charged beam generated by the beam generator; and
a detector configured to detect a charged beam reflected.

6. A charged-beam exposure method comprising:
detecting first marks provided on chips formed in a wafer, by using first charged-beam optical system, which is one of charged-beam optical systems;
calculating positions of the chips made in the wafer, from position data about the first marks detected;
detecting a second mark provided on a stage holding the wafer by using each of the charged-beam optical systems; and
adjusting position of the beam generated by each charged-beam optical system, in accordance with position data about the second mark detected.

7. The method according to claim 6, further comprising drawing a pattern by using the charged-beam optical systems based on the calculated positions of the chips.

8. The method according to claim 7, wherein the position of the beam generated by each charged-beam optical system is adjusted immediately before the charged-beam optical systems is used to draw a pattern.

9. The method according to claim 7, wherein the position of the beam generated by each charged-beam optical system is adjusted in accordance with a distance between the first charged-beam optical system and a second charged-beam optical system, and with position data about the second mark detected.
